(19) United States
(12) Patent Application Publication
(10) Pub. No.: US 2022/0395341 A1
(43) Pub. Date: Dec. 15, 2022

(54) TRAINING DEVICE AND TRAINING SYSTEM

(71) Applicant: MEDICAROID CORPORATION, Kobe-shi (JP)

(72) Inventor: Jota IDA, Kobe-shi (JP)

(73) Assignee: MEDICAROID CORPORATION, Kobe-shi (JP)

(21) Appl. No.: 17/837,079

(22) Filed: Jun. 10, 2022

(30) Foreign Application Priority Data
Jun. 14, 2021 (JP) 2021-099008

(51) Int. Cl. A61B 34/35; A61B 34/37; G06F 16/738; A61B 34/00

(52) U.S. Cl. CPC A61B 34/35; A61B 34/37; G06F 16/739; A61B 34/25; G06T 2207/20081; A61B 2034/306

(57) ABSTRACT

A training device according to an embodiment may be used for training for surgery using a surgical system that includes: a patient side apparatus including first and second robot arms; and a doctor side control apparatus including first and second input devices configured to operate the first and second robot arms respectively and a display. The training device may include: a training control unit configured to display, on the display, a first virtual surgical instrument that moves in response to operation on the first input device, and a second virtual surgical instrument that moves in response to operation on the second input device; and a storage storing therein a surgical video. The training control unit is configured to display on the display the surgical video stored in the storage with overlaying the first virtual surgical instrument and the second virtual surgical instrument on the surgical video.

### CROSS REFERENCE TO RELATED APPLICATIONS

This application claims priority based on 35 USC 119 from prior Japanese Patent Application No. 2021-099008 filed on Jun. 14, 2021, entitled “TRAINING DEVICE AND TRAINING SYSTEM”, the entire contents of which are incorporated herein by reference.

### BACKGROUND

The disclosure may relate to a training device and a training system and may particularly relate to a training device and a training system to be used for training in a surgical system including a robot arm.

In a related art, there has been known a training device that is used for training in a robotic surgical system including a robot arm (see Patent Document 1: U.S. Pat. No. 8,600,551).

Patent Document 1 discloses a simulator unit (a training device) used for training for improving skill in surgery using a robotic surgical system including a control console including an input device and a display and a manipulator configured to move surgical instruments in response to the movement of the input device. In order to execute a procedure simulated in a virtual environment, the simulator unit displays virtual surgical instruments on the display in such a manner that the displayed virtual surgical instruments move in response to the movement of the input device. The simulator unit displays on the display a plurality of selectable training menus to be performed in prepared virtual environments so that a user can select one of the training menus, and displays on the display virtual surgical instruments in a prepared virtual environment corresponding to the selected training menu, so that the user can train himself or herself in the displayed virtual environment by operating the input device.
Patent Document 1: U.S. Pat. No. 8,600,551

### SUMMARY

However, the simulator unit (the training device) disclosed in Patent Document 1 can provide only the prepared training menus to be performed in the prepared virtual environments, and thus it may be difficult for a doctor to train himself or herself for various surgeries.

An object of an embodiment of the disclosure may be to provide a training device and a training system that allows a doctor to easily train himself or herself for various surgeries.

A first aspect of the disclosure may be a training device to be used for training for surgery using a surgical system that includes: a patient side apparatus including a first robot arm to which a first surgical instrument is attached and a second robot arm to which a second surgical instrument is attached; and a doctor side control apparatus including a first input device configured to operate the first robot arm, a second input device configured to operate the second robot arm, and a display. The training device includes: a training control unit configured to display, on the display, a first virtual surgical instrument that moves in response to operation on the first input device, and a second virtual surgical instrument that moves in response to operation on the second input device; and a storage storing therein a surgical video. The training control unit is configured to display on the display the surgical video stored in the storage with overlaying the first virtual surgical instrument and the second virtual surgical instrument on the surgical video.

According to the training device according to the first aspect, the training control unit is provided that is configured to display on the display the surgical video stored in the storage with overlaying the first virtual surgical instrument and the second virtual surgical instrument on the surgical video, as described above. With this, videos that are used in the training can be generated by recording and storing actual surgical videos. Therefore, it is possible to easily generate videos of various types of surgeries. Accordingly, it is possible for a doctor (a trainee) to easily perform the training for various types of surgeries. In addition, when a proctor (an instructor) wants to train the doctor (trainee) for a specific type of surgery, the doctor (trainee) can perform pre-training of the specific type of surgery using a surgical video of the specific type of surgery performed by the proctor in advance. Further, according to the first aspect, the first virtual surgical instrument and the second virtual surgical instrument operated in the training are displayed with being overlaid on the surgical video. Therefore, the training can be easily performed even when such a surgical video is used.

A second aspect of the disclosure may be a training system that includes: a first terminal device provided in a first facility in which a first surgical system is installed, wherein the first surgical system includes: a first patient side apparatus and a first doctor side control apparatus configured to operate the first patient side apparatus; and a training control device configured to display on a first display of the first doctor side control apparatus a surgical video with overlaying a first virtual surgical instrument and a second virtual surgical instrument on the displayed surgical video; a second terminal device provided in a second facility in which a second surgical system including a second patient side apparatus and a second doctor side control apparatus configured to operate the second patient side apparatus is installed; a server provided in a third facility different from the first facility and the second facility and configured to communicate with the first terminal device and the second terminal device; and a storage device provided in the third facility and configured to store therein surgical videos. The storage device is configured to receive a first surgical video, which is a video of surgery performed using the second surgical system of the second facility, from the second terminal device and store therein the first surgical video.

According to the second aspect, the training system displays on the display the surgical video stored in the storage with overlaying the first virtual surgical instrument and the second virtual surgical instrument on the surgical video. With this, videos that are used in the training can be generated by recording and storing actual surgical videos. Therefore, it is possible to easily generate videos of various types of surgeries. Accordingly, it is possible to provide a training system that allows a doctor (a trainee) to easily perform the training for various types of surgeries. In addition, when a proctor (an instructor) wants to train the doctor (trainee) for a specific type of surgery, the doctor (trainee) can perform pre-training of the specific type of surgery using a surgical video of the specific type of surgery performed by the proctor in advance. Further, according to the second aspect, the first virtual surgical instrument and the second virtual surgical instrument operated in the training are displayed with being overlaid on the surgical video. Therefore, the training can be easily performed even when such a surgical video is used. Further, according to the second aspect, the surgical video of the surgery performed by the surgical system in the second facility can be stored in the storage device of the third facility and used in the training device of the first facility. Therefore, the training can be easily performed using the surgical video even at a place distant from the place where the surgical video is captured.

According to at least one of the aspects described above, it may be possible for a doctor to easily train himself or herself for various types of surgeries.

### BRIEF DESCRIPTION OF DRAWINGS

FIG. 1 is a diagram illustrating a view of a configuration of a surgical system according to an embodiment;

FIG. 2 is a diagram illustrating a view of a configuration of a medical manipulator according to an embodiment;

FIG. 3 is a diagram illustrating a view of a configuration of an operation handle according to an embodiment;

FIG. 4 is a diagram illustrating a view of foot pedals according to an embodiment;

FIG. 5 is a diagram illustrating a view of a configuration of an arm of the medical manipulator according to an embodiment.

FIG. 6 is a diagram illustrating a view of forceps;

FIG. 7 is a block diagram illustrating a training device according to an embodiment;

FIG. 8 is a block diagram illustrating a view of a training system according to an embodiment;

FIG. 9 is a diagram illustrating a view of a first display example (a first type surgical video or a first type surgery image) of the training device;

FIG. 10 is a diagram illustrating a view of an example of an operation screen of the training device;

FIG. 11 is a diagram illustrating a view of an example of a selection screen of the training device for selecting a surgery type and/or a surgeon;

FIG. 12 is a diagram illustrating a view of an example of a selection screen of the training device for selecting surgical instruments; and

FIG. 13 is a diagram illustrating a view of a second display example (a second type surgical video or a second type surgery image) of the training device.

### DETAILED DESCRIPTION

Descriptions are provided hereinbelow for one or more embodiments based on the drawings. In the respective drawings referenced herein, the same constituents are designated by the same reference numerals and duplicate explanation concerning the same constituents is omitted. All of the drawings are provided to illustrate the respective examples only.

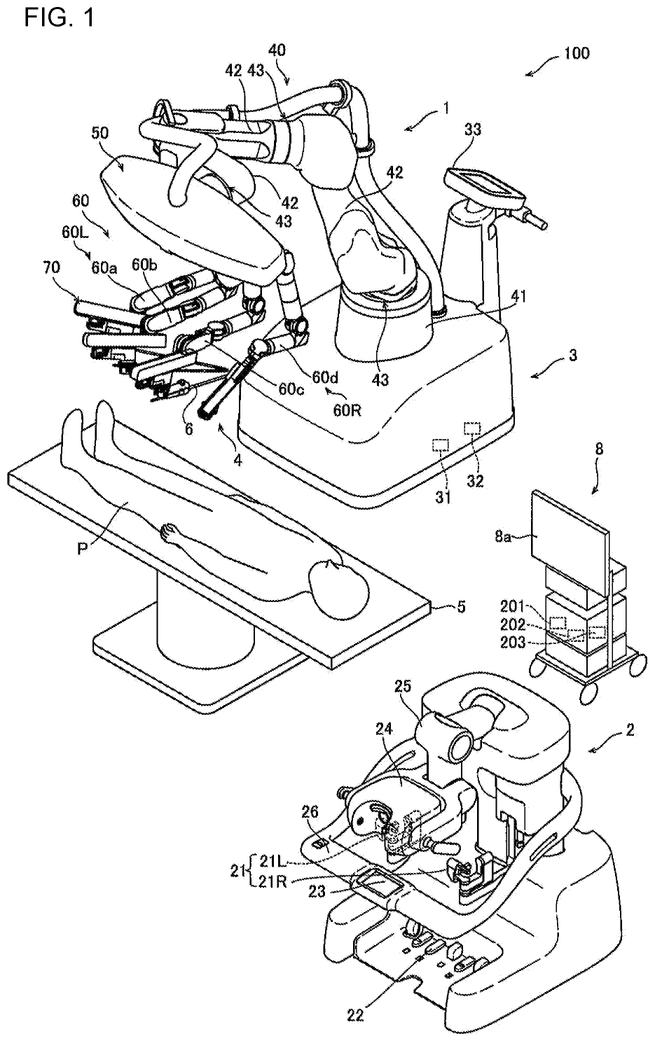

With reference to FIGS. 1 to 12, a training device 200 is explained to be used for training for surgery in a surgical system 100 according to an embodiment. The surgical system 100 includes a medical manipulator 1 serving as a patient-side apparatus and a remote control apparatus 2 serving as a doctor-side apparatus or an operator-side apparatus to operate the medical manipulator 1. The medical manipulator 1 is provided with a medical trolley 3 and is thus configured to be movable. The remote control apparatus 2 is provided at a location away from the medical manipulator 1. The medical manipulator 1 is configured to be remotely operated by the remote control apparatus 2. A surgeon (an operator) who is a doctor inputs to the remote control apparatus 2 an instruction that causes the medical manipulator 1 to perform a desired operation. The remote control apparatus 2 transmits the input instruction to the medical manipulator 1. The medical manipulator 1 operates in response to the received instruction. The medical manipulator 1 is disposed in a surgery room, as a sterile field, which is sterilized.

The remote control apparatus 2 is disposed inside the surgery room or outside the surgery room, for example. The remote control apparatus 2 includes operation handles 21, foot pedals 22, a touch panel 23, a display (or a monitor) 24, a support arm 25, and a support bar 26. The operation handles 21 are hand controllers (HC) provided for the surgeon (the operator) to input instructions.

The operation handles 21 are configured to operate the medical instruments 4. Specifically, the operation handles 21 receive an amount of movement input by the operator to operate the medical instruments 4. The operation handles 21 include an operation handle 21L, which is arranged on the left side of the surgeon (the operator) and is to be operated by the left hand of the operator O, and an operation handle 21R, which is arranged on the right side of the operator and is to be operated by the right hand of the operator. The operation handle 21L and the operation handle 21R may be examples of a “first input device” and a “second input device”, respectively.

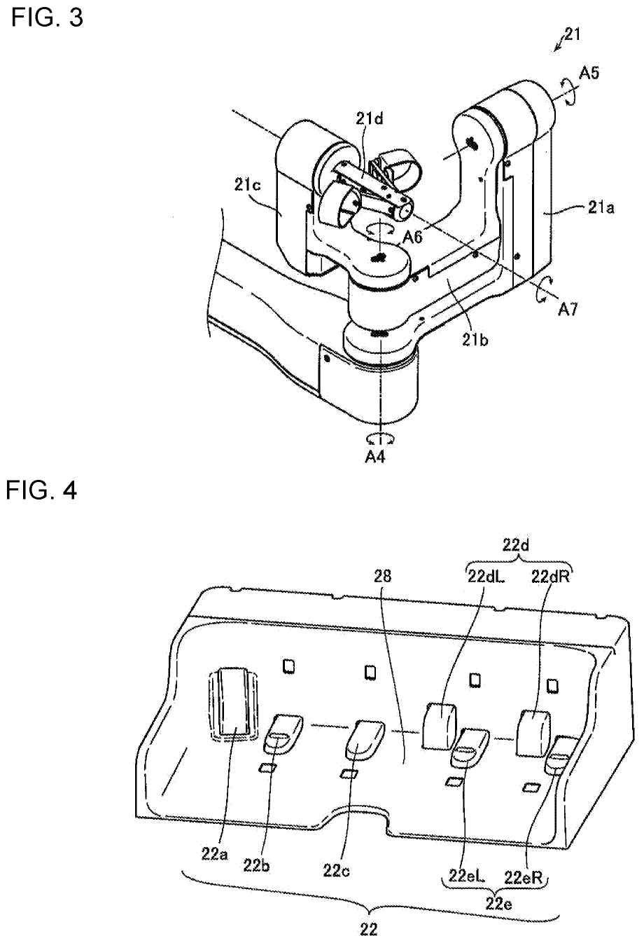

As illustrated in FIG. 3, each of the operation handles 21 includes a link portion 21a, a link portion 21b, a link portion 21c, and a link portion 21d that is to be operated by the surgeon (the operator). The link portion 21a is rotatable about an axis (joint) A4. By rotating the link portion 21a around the axis A4, the arm portion 61 described later rotates about an axis (joint) JT4. The link portion 21b is rotatable about an axis (joint) A5 with respect to the link portion 21a. By rotating the link portion 21b around the axis A5, the arm portion 61 described later rotates about an axis (joint) JT5. The link portion 21c is rotatable about an axis (joint) A6 with respect to the link portion 21b. By rotating the link portion 21c around the axis A6, the arm portion 61 rotates about an axis (joint) JT6. The link portion 21d is rotatable about an axis (joint) A7 with respect to the link portion 21c. By rotating the link portion 21d around the axis A7, the arm portion 61 rotates about an axis (joint) JT7. Note that the medical instrument 4 may be an example of a “surgical instrument”.

Further, a movement amount of the arm 60 (medical instrument 4) is scaled (changed) with respect to the operation amount received by the operation handle 21. For example, when the movement scaling ratio is set to ½, the medical instrument 4 moves ½ of the movement distance of the operation handle 21. This allows for precise fine surgery.

As illustrated in FIG. 4, the plural foot pedals 22 are provided to execute functions of the medical instrument 4. The plural foot pedals 22 are arranged on a base 28. The foot pedals 22 include a switch pedal 22a, a clutch pedal 22b, a camera pedal 22c, cutting pedals 22d, and coagulation pedals 22e. The switch pedal 22a, the clutch pedal 22b, the camera pedal 22c, the cutting pedals 22d, and the coagulation pedals 22e are operated by the foot of the operator. The cutting pedals 22d includes a cutting pedal 22dR for the right arm 60 and a cutting pedal 22dL for the left arm 60. The coagulation pedals 22e include a coagulation pedal 22eR for the right arm 60 and a coagulation pedal 22eL for the left arm 60.

The switch pedal 22a is configured to select one or more of the arms 60 that are to be operated by the operation handles 21. The clutch pedal 22b is configured to perform a clutch operation that temporarily disconnects the operational connection between the arm 60 and the operation handle 21. While the clutch pedal 22b is depressed by the operator, the operation by the operation handle 21 is not transmitted to the arm 60.

In an embodiment, the camera pedal 22c is provided for inputting a command that allows the endoscope 6 to be moved. Specifically, in response to the camera pedal 22c being depressed (stepped) by the operator, the command that allows the endoscope 6 to be moved is inputted. That is, while the command that enables the endoscope 6 to move is being inputted by the camera pedal 22c (that is, while the camera pedal 22c is depressed by the operator), the endoscope 6 is able to be moved by moving both of the operation handle 21R and operation handle 21L.

While the cutting pedal 22d (coagulation pedal 22e) is depressed (stepped) by the operator, an electrosurgical device is activated.

As illustrated in FIG. 1, the display 24 is a display device of a scope type configured to display an image captured by the endoscope 6. The support arm 25 supports the display 24 in such a manner that the height of the display 24 is adjusted to the height of the face of the operator. The touch panel 23 is disposed on the support bar 26. When a sensor(s) provided in the vicinity of the display 24 detects the head of the operator, the medical manipulator 1 can be operated by the remote control apparatus 2. The operator operates the operation handles 21 and the foot pedals 22, while viewing the surgical site (or affected area) displayed on the display 24. With this, the instruction is input to the remote control apparatus 2. The instruction that is input to the remote control apparatus 2 is transmitted to the medical manipulator 1.

The medical trolley 3 is provided with a control unit 31 (circuitry and/or one or more processors) that controls the operation of the medical manipulator 1 and a storage 32 that stores therein programs for controlling the operation of the medical manipulator 1. Based on the instruction inputted to the remote control apparatus 2, the control unit 31 of the medical trolley 3 controls the operation of the medical manipulator 1.

Further, the medical trolley 3 is provided with an input device 33. The input device 33 is configured to accept operations to move or change posture of a positioner 40, an arm base 50, and arms 60, mainly to prepare for surgery before the surgery.

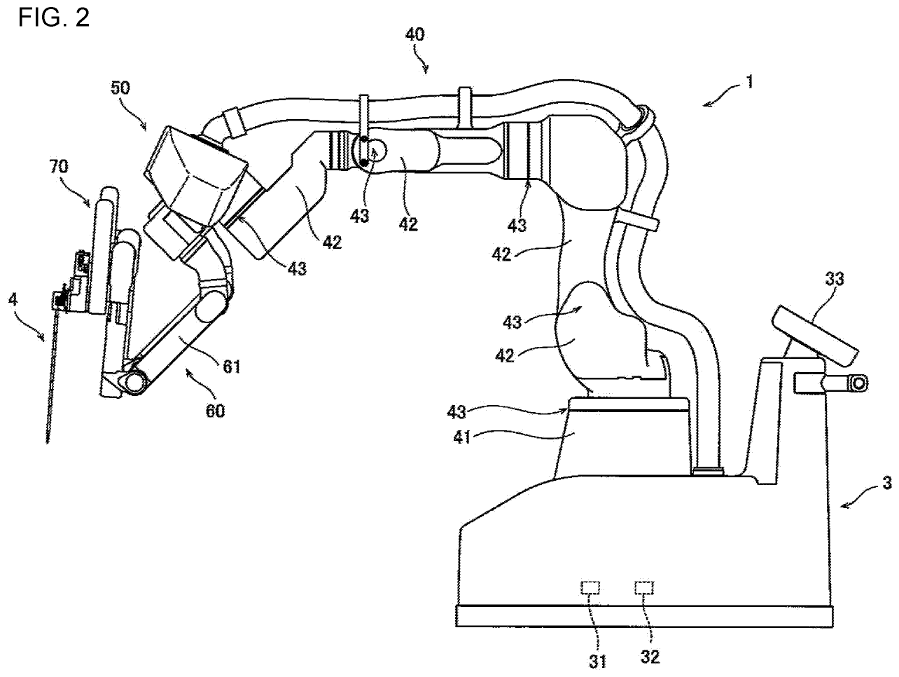

As illustrated in FIGS. 1 and 2, the medical manipulator 1 is disposed in the surgery room. The medical manipulator 1 includes the medical trolley 3, the positioner 40, the arm base 50, and the arms 60. The arm base 50 is attached to a distal end of the positioner 40. The arm base 50 is a relatively long rod shape (elongate shape). Base portions (proximal end portions) of the arms 60 are attached to the arm base 50. Each of the arms 60 is configured such that the arm 60 is able to take a folded posture (storage posture). The arm base 50 and the arms 60 are used with being covered with a sterile drape (not illustrated). The arm 60 supports the medical instrument 4.

The positioner 40 is configured as a 7-axis articulated robot. The positioner 40 is disposed on the medical trolley 3. The positioner 40 is configured to move the arm base 50. Specifically, the positioner 40 is configured to move the position of the arm base 50 three-dimensionally.

The positioner 40 includes a base portion 41 and link portions 42 connected to the base portion 41. The link portions 42 are connected to each other via joints 43.

As illustrated in FIG. 1, to the distal end of each of the arms 60, the medical instrument 4 is attached. The medical instruments 4 include, for example, instruments that are replaceable, an endoscope 6 configured to image a surgical site (that is, to capture an image of a surgical site), and the like.

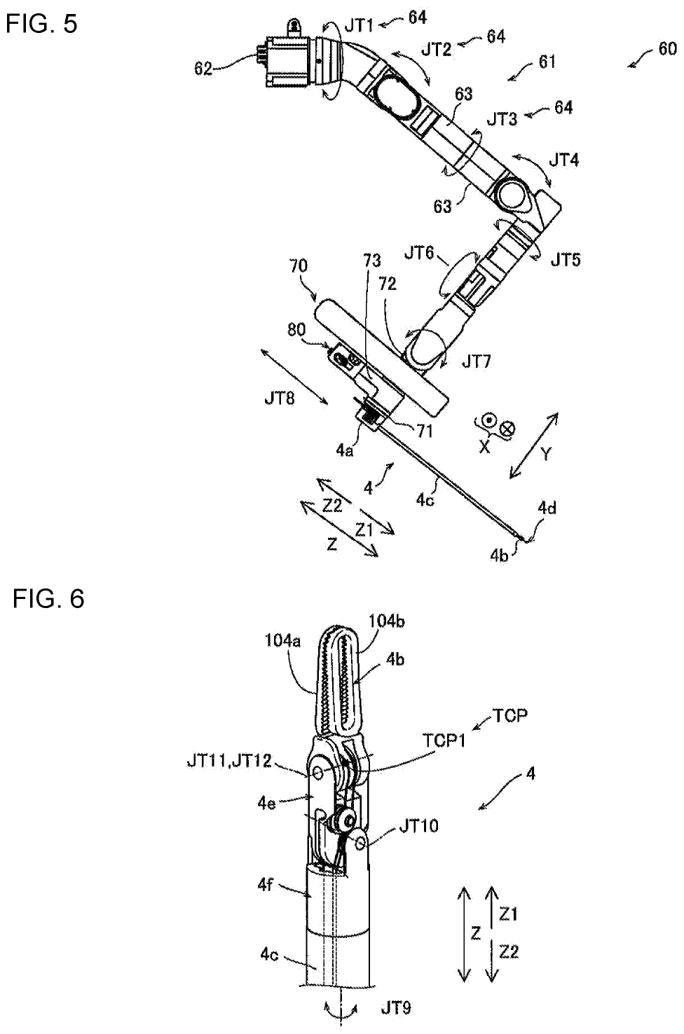

As illustrated in FIG. 5, the instrument is provided with a driven unit 4a, which is driven by servomotors provided in a holder 71 of the arm 60. To the distal end of the instrument, forceps 4b as an end effector is provided.

As illustrated in FIG. 6, the instrument includes: a first support 4e having a distal end portion thereof that rotatably supports proximal end portions of end effector members 104a and 104b about an axis (joint) JT11; a second support 4f having a distal end portion thereof that rotatably supports a proximal end portion of the first support 4e about an axis (joint) JT10; and a shaft 4c connected a proximal end portion of the second support 4f. The driven unit 4a, the shaft 4c, the second support 4f, the first support 4e, and the forceps 4b are arranged along the Z direction. The axis JT11 is orthogonal to a direction (Z direction) in which the shaft 4c extends. The axis JT10 is provided away from the axis JT11 in the direction in which the shaft 4c extends, and is orthogonal to the axis JT11 and orthogonal to the direction in which the shaft 4c extends.

The forceps 4b is attached to the first support 4e so as to be rotatable about the axis JT11. The second support 4f rotatably supports the first support 4e about the axis JT10. In other words, the first support 4e is attached to the second support 4f so as to be rotatable about the axis JT10. A distal side (Z1 side) portion of the first support 4e a has a U-shape. A tool center point (TCP1, Clevis) is set at the center of the U-shaped distal side portion of the first support 4e along the axis JT11.

The medical instrument 4 (forceps 4b) includes an axis (joint) JT9 as a rotation axis of the shaft 4c (extending along the direction in which the shaft 4c extends) and an axis (joint) JT12 about which the forceps 4b open and close. Note that the plural (for example, four) the servomotors M2 are provided in the holder 71 of the arm 60 and rotors (rotation members) in the driven unit 4a are driven by the plural servomotors M2. As a result, the medical instrument 4 is driven about the J9 axis to the J12 axis.

Next, a configuration of the arm 60 is described in detail.

As illustrated in FIG. 5, the arm 60 includes an arm portion 61 (the base portion 62, the link portions 63, the joint portions 64) and a translation movement mechanism 70 provided at the distal end portion of the arm portion 61. The arm 60 is configured such that the distal end portion thereof is three-dimensionally movable with respect to the proximal side (the arm base 50) of the arm 60. The arm portion 61 is configured as a 7-axis articulated robot arm. The plural arms 60 have the same configuration as each other.

As illustrated in FIG. 5, the arm 60 includes the axis (joints) JT1 to JT7 as rotation axes and an axis (joint) J8 as a linear motion axis. The axes JT1 to JT7 correspond to the rotation axes of the joint portions 64 of the arm portion 61. The axis JT7 corresponds to the proximal end side link portion 72 of the translational movement mechanism 70. An axis (joint) JT8 is an axis for moving the distal end side link portion 73 of the translational movement mechanism 70 relative to the proximal end side link portion 72 along the Z direction.

The translation movement mechanism 70 is provided on a side of the distal end of the arm portion 61. The medical instrument 4 is attached to the translation movement mechanism 70. The translation movement mechanism 70 translationally moves the medical instrument 4 in the insertion direction of the medical instrument 4 into a patient P. The translation movement mechanism 70 is configured to translationally move the medical instrument 4 relative to the arm portion 61. Specifically, the translation movement mechanism 70 is provided with the holder 71 configured to hold the medical instrument 4. The holder 71 accommodates therein the servomotors.

As illustrated in FIG. 1, the endoscope 6 is attached to one (for example, the arm 60c) of the plural arms 60, and the medical instruments 4 other than the endoscope 6 are attached to the other arms 60 (for example, the arms 60a, 60b, and 60d). Specifically, for surgery, the endoscope 6 is attached to one of the four arms 60, and the medical instruments 4 (forceps 4b, etc.) other than the endoscope 6 are attached to the other three arms 60.

Further, the number of arms 60 that can be operated by the operation handles 21 is two. For example, the operation handle 21L operates the left arm 60L (for example, the arm 60a) that supports the surgical instrument 4L. The operation handle 21R operates the right arm 60R (for example, the arm 60d) that supports the surgical instrument 4R. Note that the right arm 60R (arm 60d) may be an example of a “first robot arm.” Note that the left arm 60L (arm 60a) may be an example of a “second robot arm.” The surgical instrument 4R and surgical instrument 4L may be examples of a “first surgical instrument” and a “second surgical instrument”.

The surgical system 100 is provided with a monitor cart 8 as illustrated in FIG. 1. The monitor cart 8 includes a display 8a. The display 8a of the monitor cart 8 displays the image same as the image displayed in the display 24 of the remote control apparatus 2. That is, the image same as the image that is displayed on the display 24 and viewed by the surgeon can viewed by the worker (nurse, assistant, etc.) around the medical manipulator 1 and the patient P by means of the display 8a of the monitor cart 8.

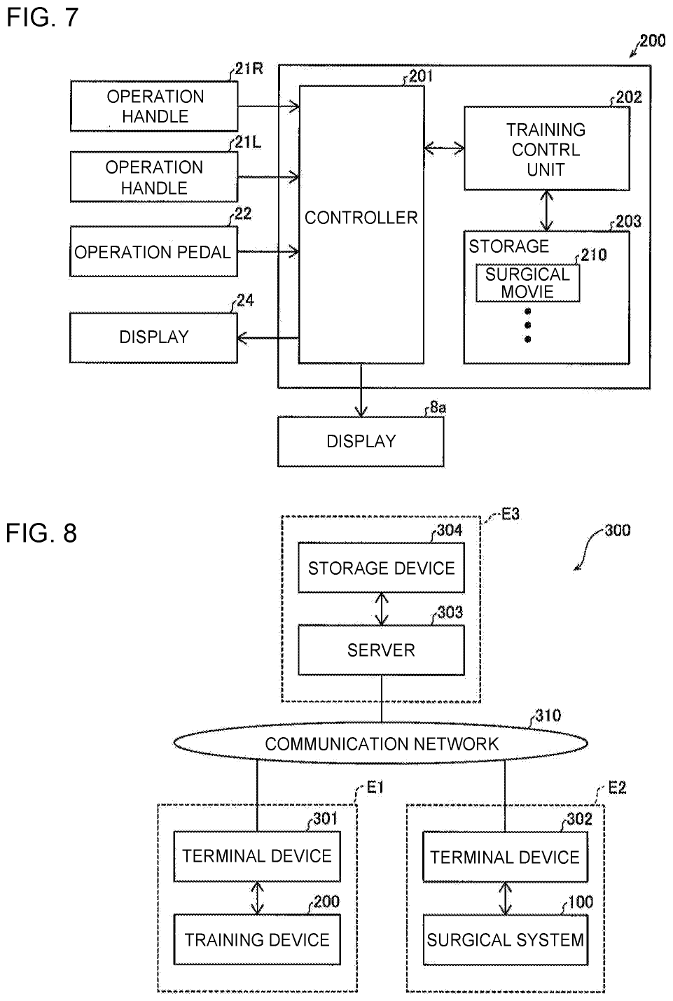

As illustrated in FIG. 7, the training device 200 includes a control unit 201, a training control unit 202 (a training control device 202), and a storage 203 or a memory. For example, as illustrated in FIG. 1, the control unit 201, the training control unit 202, and the storage 203 are provided in the monitor cart 8. The training device 200 is used for training for surgery using the surgical system 100. That is, the training device 200 is used for training for how to operate the surgical system 100 with receiving the operations of the operation handles 21 and the foot pedals 22. Note that the control unit 201 and the training unit 202 includes circuitry and/or one or more processors configured with programs to perform desired operations and functions.

As illustrated in FIG. 7, the control unit 201 receives the operation of the operation handles 21 (21R and 21L) and the operation of the foot pedals 22 of the remote control apparatus 2 of the surgical system 100 so as to control the operation of the medical manipulator 1. The control unit 201 transmits the image captured by the endoscope 6 to the display 24. That is, the control unit 201 is also used to operate the surgical system 100 during actual surgery. The training is performed using the operation handles 21R and 21L of the remote control apparatus 2, which are to be used at the time of actual surgery to operate the medical manipulator 1.

Further, the control unit 201 receives the operation of the operation handles 21 (21R and 21L) of the remote control apparatus 2 and the operation of the foot pedals 22 at the time of training, and transmits information of the operations thereof to the training control unit 202. Further, the control unit 201 transmits to the display 24 the image to be used for training that is transmitted from the training control unit 202. Further, the control unit 201 transmits to the display 8a the image to be used for training that is transmitted from the training control unit 202.

The storage 203 stores therein one or more surgical videos 210 each of which is recorded (captured) during surgery of the surgical site where the surgical instrument 4R and the surgical instrument 4L operates. For example, the storage 203 stores a plurality of surgical videos 210.

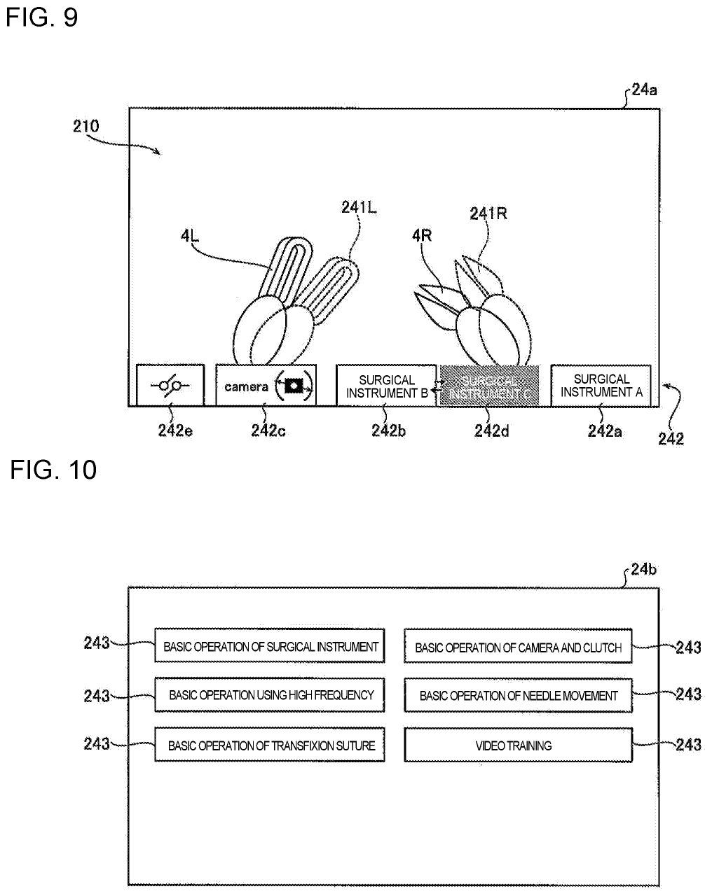

Here, in an embodiment, as illustrated in FIG. 9, the training control unit 202 is configured to display on the display 24 a virtual surgical instrument 241R that moves in correspondence with the operation by the operation handle 21R and a virtual surgical instrument 241L that moves in correspondence with the operation by the operation handle 21L. Further, the training control unit 202 is configured to display on the display 24 the surgical video 210 stored in the storage 203 with overlaying the virtual surgical instruments 241R and 241L on the displayed surgical video 210. The virtual surgical instrument 241R may be an example of a “first virtual surgical instrument” and the virtual surgical instrument 241L may be an example of a “second virtual surgical instrument.”

In the training using the training device 200, the operator performs training by operating the operation handles 21R and 21L to make the virtual surgical instruments 241R and 241L that are moved in response to the operation handles follow (overlap with) the displayed surgical instruments 4R and 4L in the surgical video 210, respectively. That is, the operator (a doctor who is a trainee) performs the training by tracing the operation performed by a proctor (a doctor who is a trainer or an instructor) in the surgical video of the surgery performed by the proctor while watching the surgical video 210.

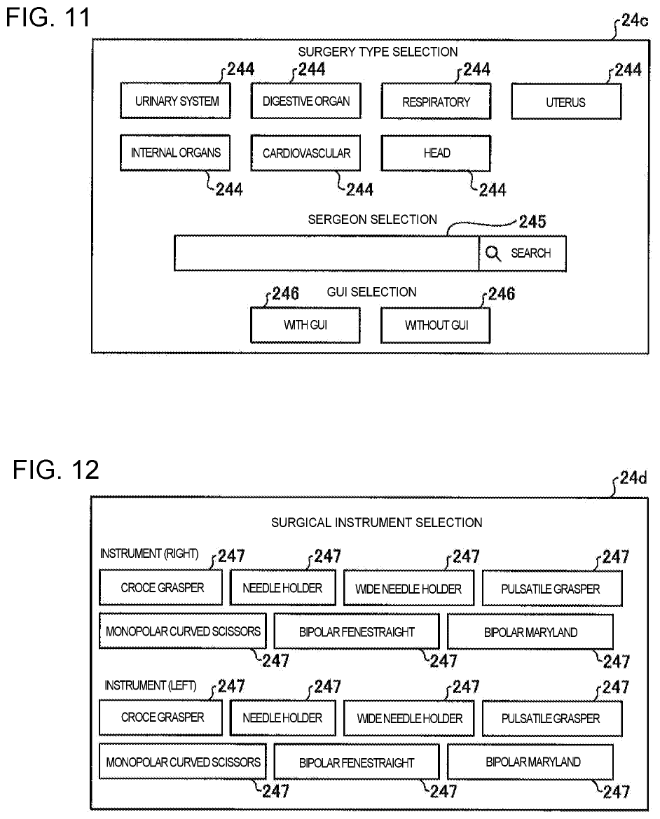

As illustrated in FIGS. 10 and 11, the training control unit 202 is configured to display on the display 24 selection screens 24b and 24c for selecting one from the plurality of surgical videos 210.

As illustrated in FIG. 10, the selection screen 24b is an example of a standard exercise home screen for training. On the standard exercise home screen, a plurality of buttons 243 for plurality of training menus are displayed. The operator selects one of the plurality of training menus (the plurality of buttons 243), by operating the operation handles 21R and 21L or the touch panel 23.

As illustrated in FIG. 11, the training control unit 202 is configured to display on the display 24 the selection screen 24c through which the operator can select one of the surgical videos 210 based on at least one of a surgery type and a name of a surgeon who performed the surgery recorded in the surgical video. The selection screen 24c is an example of a screen for selecting a surgery type or a surgeon under which the operator would like to perform training. The selection screen 24c displays a plurality of buttons 244 for selecting one of the types of surgery (for example, urinary system, digestive organ, respiratory, uterus, internal organs, cardiovascular, head, or the like) and a search box 245 for inputting a name of a surgeon so as to search one or more of videos 210 of surgeries performed by the surgeon. The operator operates the operation handles 21R and 21L or the touch panel 23 to input and/or select a surgery type or a name of a surgeon. Note that the operator may input and/or select a surgery type or a name of a surgeon by using voice input. Further, the selection screen 24c displays buttons 246 for selecting a type of the surgical video 210 between a first type surgical video (with GUI) which includes a graphical user interface and a second type surgical video (without GUI) which does not includes the graphical user interface.

The storage 203 stores the two types of surgical videos, one of which is the first type which includes the graphical user interface (for example, a recorded graphical user interface) displaying information about the surgical instrument 4R and information about the surgical instrument 4L in the surgery when the surgical video is recorded, and the other of which is the second type which does not include the graphical user interface. The surgical video of the first type and the surgical video of the second type are the same in the surgical video captured by the endoscope, except for presence or absence of the graphical user interface which displays the information about the surgical instruments 4R and 4L.

When the “with GUI” button 246 for displaying GUI is selected through the selection screen 24c, the training control unit 202 displays on the display 24 the first type surgical video which includes the graphical user interface. On the other hand, when the “without GUI” button 246 for displaying no GUI is selected through the selection screen 24c, the training control unit 202 displays on the display 24 the second type surgical video which does not include the graphical user interface. At that time, the training control unit 202 generates a graphical user interface displaying information about the virtual surgical instruments 241R and 241L, and superimposes the generated graphical user interface for the virtual surgical instruments 241R and 241L on the second type surgical video on the display 24.

As illustrated in FIG. 12, the training control unit 202 displays, on the display 24, a setting screen 24d that allows to select one surgical instrument from plural types of surgical instruments as the first virtual surgical instrument 241R and to select one surgical instrument from the plural types of surgical instruments as the second virtual surgical instrument 241L. The setting screen 24d displays plural buttons 247 for selecting one of the surgical instrument types for each of the virtual surgical instruments 241R and 241L. The operator operates the operation handles 21R and 21L or the touch panel 23 to select one of the surgical instrument types of each of the virtual surgical instruments 241R and 241L. Based on the selected surgical instrument types, the types of the virtual surgical instruments 241R and 241L which will be displayed on the display 24 in the training are changed.

As illustrated in FIG. 9, the training control unit 202 overlays the virtual surgical instruments 241R and 241L on the surgical video 210 in such a manner that the virtual surgical instruments 241R and 241L are displayed in light (thin) color or translucent on the surgical video 210.

First, a case where the first type surgical video including the graphical user interface is selected through the selection screen 24c will be described.

As illustrated in FIG. 9, the surgical video 210 includes a graphical user interface 242 that displays information about the surgical instrument 4R and information about the surgical instrument 4L during the surgery recorded in the surgical video 210. That is, the surgical video 210 includes, in addition to the image (the video image) captured by the endoscope 6 during the surgery, the graphical user interface 242 displaying the information about the surgical instruments 4R and 4L operated in the surgery.

The graphical user interface 242 includes a first area 242a indicating information about the surgical instrument 4R to be operated by the right hand operation handle 21R, a second area 242b indicating information about the surgical instrument 4L to be operated by the left hand operation handle 21L, and a third area 242c indicating information about the endoscope 6. The graphical user interface 242 also includes a fourth area 242d that indicates information about a replacement surgical instrument. The graphical user interface 242 also includes a fifth area 242e that indicates an operating state of the clutch pedal 22b.

In the first area 242a, the type of the first surgical instrument 4R is displayed. In the second area 242b, the type of the second surgical instrument 4L is displayed. Further, when the foot pedal 22 (the incision pedal 22dR or the coagulation pedal 22eR) for the surgical instrument 4R is operated, the first area 248a is displayed in a color corresponding to the operation. For example, the first area 242a is displayed (highlighted) in yellow when the incision pedal 22dR is operated. Also, the first area 242a is displayed (highlighted) in blue when the coagulation pedal 22eR is operated.

When the foot pedal 22 (the cutting pedal 22dL or the coagulation pedal 22eL) for the surgical instrument 4L is operated, the second area 242b is displayed in a color corresponding to the operation. For example, the second area 242b is displayed (highlighted) in yellow when the cutting pedal 22dL is operated. To the contrary, the the second area 242b is displayed (highlighted) in blue when the coagulation pedal 22eL is operated.

When the foot pedal 22 (the camera pedal 22c) for the endoscope 6 is operated, the third area 242c is displayed in a color corresponding to the operation. For example, when the camera pedal 22c is operated, the background color and the text color of the third area 242c are inverted.

The fourth area 242d displays the type of the replacement surgical instrument. The information in the fourth area 242d is displayed with paler color than the information in the first area 242a and the second area 242b.

An image (video image) captured by the endoscope 6 during the surgery is recorded as the surgical video 210 of the second type, and the image (video image) captured by the endoscope 6 during the surgery and the graphical user interface 242 as described above during the surgery are combined and recorded as the surgical video 210 of the first type. In addition, the surgical video 210 may be recorded together with the voice of the surgeon during the surgery.

Then, at the time of training, the training control unit 202 overlays the virtual surgical instruments 241R and 241L on the surgical video 210 (the first type surgical video) including the graphical user interface 242.

The training control unit 202 recognizes the operation of the foot pedals 22 in the surgery based on the graphical user interface 242 in the surgical video 210. The training control unit 202 acquires the operator's operation of the foot pedals 22 during the training. The training control unit 202 may determine whether or not the foot pedals 22 are operated at appropriate timings in the training by comparing the operation of the foot pedals 22 during the surgery in the recorded surgical video with the operation of the foot pedals 22 during the training.

The training control unit 202 may automatically sets the types of the virtual surgical instruments 241R and 241L by recognizing the types of the surgical instruments 4R and 4L in the surgical video 210 based on the graphical user interface 242 of the surgical video 210.

Next, a case where the second type surgical video which does not include the graphical user interface is selected through the selection screen 24c will be described.

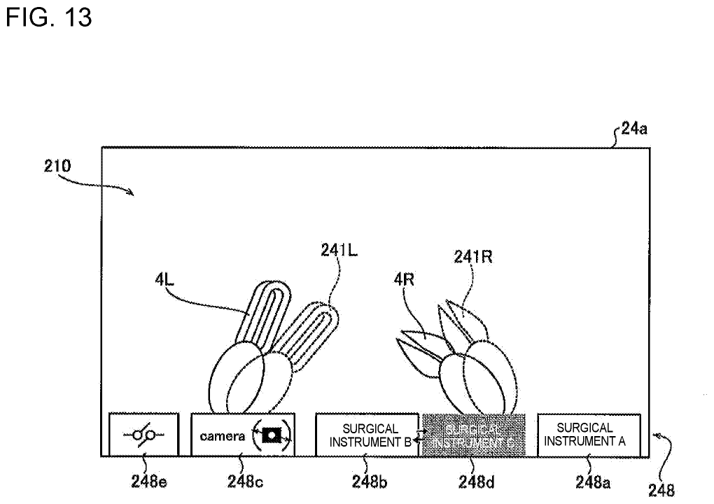

As illustrated in FIG. 13, when the second type surgical video is selected through the selection screen 24c, the surgical video 210 does not include the graphical user interface 242 recorded in the surgery but the training control unit 202 displays on the display 24 the surgical video 210 with overlaying on the displayed surgical video 210 a graphical user interface 248 that indicates information about the virtual surgical instrument 241R and information about the virtual surgical instrument 241L. That is, the surgical video 210 includes only a video that is captured by the endoscope 6 during the surgery, and the training control unit 202 generates the graphical user interface 248 displaying information on the virtual surgical instruments 241R and 241L operated during the training and superimposes the generated graphical user interface 248 during the training on the surgical video 210 (the second type surgical video) on the display 24.

The graphical user interface 248 includes a first area 248a indicating information about the virtual surgical instrument 241R to be operated by the right hand operation handle 21R, a second area 248b indicating information about the virtual surgical instrument 241L to be operated by the left hand operation handle 21L, and a third area 248c indicating information about the endoscope 6. The graphical user interface 248 also includes a fourth area 248d that provides information about the replacement surgical instrument. Further, the graphical user interface 248 includes a fifth area 248e indicating an operating state of the clutch pedal 22b.

In the first area 248a, the type of the first virtual surgical instrument 241R is displayed. In the second area 248b, the type of the second virtual surgical instrument 241L is displayed. Further, when the foot pedal 22 (the incision pedal 22dR or the coagulation pedal 22eR) for the virtual surgical instrument 241R is operated, the first area 248a is displayed in a color corresponding to the operation. For example, the first area 248a is displayed (highlighted) in yellow when the incision pedal 22dR is operated. Also, the first area 248a is displayed (highlighted) in blue when the coagulation pedal 22eR is operated.

When the foot pedal 22 (the cutting pedal 22dL or the coagulation pedal 22eL) for the virtual surgical instrument 241L is operated, the second area 248b is displayed in a color corresponding to the operation. For example, the second area 248b is displayed (highlighted) in yellow when the cutting pedal 22dL is operated. To the contrary, the the second area 248b is displayed (highlighted) in blue when the coagulation pedal 22eL is operated.

When the foot pedal 22 (the camera pedal 22c) for the endoscope 6 is operated, the third area 248c is displayed in a color corresponding to the operation. For example, when the camera pedal 22c is operated, the background color and the text color of the third area 248c are inverted.

The fourth area 248d displays the type of the replacement surgical instrument. The information in the fourth area 248d is displayed with paler color than the information in the first area 248a and the second area 248b.

In the training, the training control unit 202 overlays the virtual surgical instruments 241R and 241L and the graphical user interface 248 on the surgical video 210 (the second type surgical video) which does not include the graphical user interface 242.

Further, when the second type surgical video 210 is played (displayed) on the display 24, the training control unit 202 recognizes the operation of the foot pedals 22 in the surgery based on the graphical user interface 242 in the first type surgical video 210 corresponding to the displayed second type surgical video 210. That is, the first type surgical video is not displayed on the display 24, but is used for recognizing the timings of the operation of the foot pedals 22 during the surgery. The training control unit 202 also acquires the operator's operation of the foot pedals 22 during the training. The training control unit 202 may determine whether or not the foot pedals 22 are operated at appropriate timings in the training by comparing the operation of the foot pedals 22 during the surgery in the recorded surgical video with the operation of the foot pedals 22 during the training.

As illustrated in FIG. 8, the training system 300 includes a server 303 and a storage device 304 (a storage). The server 303 is installed in a third facility E3, which is different from a first facility E1 and a second facility E2, such that the server 303 is communicable with a terminal device 301 in the first facility E1 in which the training device 200 is installed and a terminal device 302 in the second facility E2 in which the surgical system 100 is installed. The storage device 304 stores therein the surgical videos 210 each of which is recorded during the surgery performed using the surgical system 100 of the second facility E2. The storage device 304 is provided in, for example, the third facility E3. The terminal device 301 may be an example of a “first terminal device” and the terminal device 302 may be an example of a “second terminal device.”

The terminal device 301 and the server 303 can communicate with each other via a communication network 310. The terminal device 302 and the server 303 can communicate with each other via the communication network 310.

In response to an upload request to upload the surgical video 210 of the surgery performed using the surgical system 100 of the second facility E2, the server 303 receives the surgical video 210 from the terminal device 302 of the second facility and stores the surgical video 210 in the storage device 304 of the third facility.

In response to a download request from the terminal device 301 to download the surgical video, the server 303 permits the terminal device 301 to download the surgical video stored in the storage device 304.

For example, in the surgical video 210 of the surgery performed by the proctor using the surgical system 100 at the second facility E2, a name of the proctor is registered as a surgeon. In addition, the surgeon causes the server 303 to upload the surgical video 210 in order to allow doctors (trainees) to use the surgical video 210. In this case, the range in which the surgical video 210 can be downloaded can be limited to the trainees. In addition, a usage fee may be incurred for the use of the surgical video 210. In such a case, the usage fee is managed by the server 303. That is, the server 303 manages the collection of the usage fees from the users and manages the payment to the surgeon of the surgical video 210. That is, the payment may be made to the surgeon of the surgical video 210 according to the download status (such as the number of downloads or the like).

(Effects)

According to one or more embodiments, effects as described below can be obtained.

In an embodiment described above, the training control unit 202 is provided which is configured to display on the display 24 the surgical video 210 stored in the storage 203 with overlaying the virtual surgical instruments 241R and 241L on the surgical video 210. With this configuration, videos to be used for the training can be generated by recording and storing actual surgical videos. Therefore, it is possible to easily generate the surgical videos 210 on various types of surgeries for the training. Accordingly, it is possible for a doctor to easily perform the training for the various types of surgeries. In addition, when the proctor (instructor) wants to train the doctor (trainee) for a specific type of surgery, the doctor (trainee) can perform pre-training of the specific type of surgery using the surgical video 210 of the specific type of surgery performed by the proctor. Further, in an embodiment described above, the virtual surgical instruments 241R and 241L operated in the training are displayed with being overlaid on the surgical video 210. Therefore, the training can be easily performed even when such a surgical video 210 is used.

Further, in an embodiment described above, the storage 203 stores the plural surgical videos 210 and the training control unit 202 is configured to display on the display 24 the selection screen 24c for selecting one surgical video 210 from the plural surgical videos 210. Accordingly, the surgical video 210 suitable for the training can be selected from the plural surgical videos 210 through the selection screen 24c, so that the training for the desired surgery can be easily performed.

In an embodiment described above, the training control unit 202 is configured to display on the display 24 the selection screen 24c through which the operator can select one of the surgical videos 210 based on which type of the surgery and/or who (surgeon) performed the surgery. Therefore, it possible to easily select the surgical video 210 corresponding to a specific type of the surgery for the training or corresponding to the surgery performed by a specific proctor. Thus, the doctor (trainee) who wants to train himself or herself can easily select the surgical video 210 suitable for the training.

In an embodiment described above, the training control unit 202 is configured to display, on the display 24, the setting screen 24d that prompts to select one among the plural types of surgical instruments as the first virtual surgical instrument 241R and to select one among the plural types of surgical instruments as the second virtual surgical instrument 241L. Therefore, the surgical instruments whose types are the same as those of the surgical instruments 4R and 4L in the surgical video 210 can be set as the virtual surgical instruments 241R and 241L, respectively. Thus, the training can be performed by operating the virtual surgical instruments 241R and 241L overlaid on the surgical video 210 to make the virtual surgical instruments 241R and 241L overlap with (follow) the surgical instruments 4R and 4L in the surgical video 210, respectively.

In an embodiment described above, the training control unit 202 is configured to overlay the virtual surgical instruments 241R and 241L on the surgical video 210 in such a manner that the virtual surgical instruments 241R and 241L are displayed in lighter (thinner, paler) color or translucent on the surgical video 210. Therefore, it is possible to prevent the surgical video 210 from becoming difficult to see during the training due to the overlaid virtual surgical instruments 241R and 241L. In addition, the virtual surgical instruments 241R and 241L can be easily distinguished from the surgical instruments 4R and 4L.

Further, in an embodiment described above, the surgical video 210 (first type surgical video) includes the graphical user interface 242 that displays the information about the surgical instrument 4R and the information about the surgical instrument 4L at the time of the surgery recorded in the surgical vide 210, and the training control unit 202 is configured to overlay the virtual surgical instruments 241R and 241L on the surgical video 210 (first type surgical video) which includes the graphical user interface 242. Therefore, the information on the surgical instrument 4R and the information on the surgical instrument 4L when the surgical video 210 is captured can also be checked (viewed) during the training. Thus, the training can be performed more appropriately while checking the states of the surgical instruments 4R and instrument 4L during the surgery.

Further, in an embodiment described above, the surgical video 210 (second type surgical video) does not include the graphical user interface 242 that displays the information about surgical instrument 4R and the information about surgical instrument 4L at the time of the surgery recorded in the surgical vide 210, and the training control unit 202 is configured to overlay the virtual surgical instruments 241R and 241L and the graphical user interface 248 on the surgical video 210 (the second type surgical video) which does not include the graphical user interface 242. Therefore, the training that imitates an actual surgery can be done by only following the movements of the surgical instruments 4R and 4L in the surgical video 210. Further, the training can be done using both of the first type surgical video 210 and the second type surgical video 210 performed by the same surgeon (proctor). Therefore, more fulfilling training can be done.

In an embodiment described above, the training is performed using the operation handles 21R and 21L of the remote control apparatus 2, which are used to operate the medical manipulator 1 at the time of the surgery. Therefore, the training can be done using the remote control apparatus 2 to be used at the time of the surgery, so that it is not necessary to separately provide a remote control apparatus 2 dedicated for such a training.

In an embodiment described above, the server 303 is configured, in response to the request to upload the surgical video 210 of the surgery performed using the surgical system 100 in the second facility E2, to receive the surgical video 210 from the terminal device 302 in the second facility E2 and store the surgical video 210 in the storage device 304. Therefore, the surgical video 210 of the surgery performed by the proctor (instructor) at the second facility E2 and uploaded to the storage device 304 can be downloaded to the training device 200 in the first facility E1. Thus, the doctor (trainee) can perform the training at the first facility E1 different from the second facility E2.

In an embodiment described above, the server 303 is configured, in response to the download request from the terminal device 301 in the first facility E1 to download the surgical video, to permit to download the surgical video stored in the storage device 304. As a result, by downloading the surgical video stored in the storage device 304, the training can be easily performed at the first facility E1 using the downloaded surgical video 210.

### Modifications

Note that one or more embodiments disclosed herein should be considered as exemplary in all respects and do not limit the invention. The scope of the invention is indicated by claims, not by explanation of one or more embodiments described above, and includes equivalents to the claims and all alterations (modification) within the same.

For example, in one or more embodiments described above, the case has been described in which the training is performed using the first input device and the second input device of the doctor side control apparatus, which is to be used at the time of surgery to operate the patient side apparatus. However, the invention is not limited thereto. In the invention, a first input device and a second input device used for the training may be provided separately from the doctor side control apparatus, which is to be used at the time of surgery.

Further, in one or more embodiments described above, the case has been described in which the graphical user interface is recorded in the surgical video together with the image captured by the endoscope during the surgery. However, the invention is not limited thereto. For example, the graphical user interface may not be recorded in the surgical video. In such a case, the surgical video which is captured by the endoscope during the surgery may be overlaid with a graphical user interface that indicates the state during the training.

In one or more embodiments described above, the case has been described in which the patient side apparatus is provided with the medical instrument for replacement. However, the disclosure is not limited thereto. For example, the patient side apparatus is not provided with such a medical instrument for replacement.

In one or more embodiments described above, the case has been described in which the number of the robot arms provided is four. However, the invention is not limited thereto. The number of the robot arms may be any number as long as at least two are provided.

In one or more embodiments described above, the case has been described in which each of the robot arms and the positioner are configured as the 7-axis articulated robot. However, the invention is not limited thereto. For example, each of the robot arms and the positioner may be configured as an articulated robot other than the 7-axis articulated robot (for example, a 6-axis articulated robot, an 8-axis articulated robot, or the like).

Further, in one or more embodiments described above, the case has been described in which the patient side apparatus includes the medical trolley, the positioner, and the arm base. However, the invention is not limited thereto. For example, the patient side apparatus may include only robot arms and not include the medical trolley, the positioner, and the arm base.

The invention includes other embodiments or modifications in addition to one or more embodiments and modifications described above without departing from the spirit of the invention. The one or more embodiments and modifications described herein are to be considered in all respects as illustrative, and not restrictive. The scope of the invention is indicated by the appended claims rather than by the foregoing description. Hence, all configurations including the meaning and range within equivalent arrangements of the claims are intended to be embraced in the invention.

1. A training device to be used for training for surgery using a surgical system that comprises: a patient side apparatus including a first robot arm to which a first surgical instrument is attached and a second robot arm to which a second surgical instrument is attached; and a doctor side control apparatus including a first input device configured to operate the first robot arm, a second input device configured to operate the second robot arm, and a display, the training device comprising:
a training control unit configured to display, on the display, a first virtual surgical instrument that moves in response to operation on the first input device, and a second virtual surgical instrument that moves in response to operation on the second input device; and
a storage storing therein a surgical video, wherein
the training control unit is configured to display on the display the surgical video stored in the storage with overlaying the first virtual surgical instrument and the second virtual surgical instrument on the surgical video.

2. The training device according to claim 1, wherein
the storage stores therein a plurality of surgical videos,
the training device is configured to display on the display a selection screen that allows to select one from the plurality of surgical videos.

3. The training device according to claim 2, wherein
the training control unit is configured to display on the display the selection screen that allows to select the one from the plurality of surgical videos based on at least one of a type of the surgery recorded in the surgical video and a doctor who performed the surgery in the surgical video.

4. The training device according to claim 1, wherein
the training control unit is configured to display, on the display, a setting screen that allows to select one surgical instrument from a plurality of types of surgical instruments as the first virtual surgical instrument and to select one surgical instrument from a plurality of types of surgical instruments as the second virtual surgical instrument.

5. The training device according to claim 1, wherein
the training control unit is configured to overlay the first virtual surgical instrument and the second virtual surgical instrument that are displayed in a thin color or translucent on the surgical video.

6. The training device according to claim 1, wherein
the surgical video is a surgical video of a first type which includes a graphical user interface that indicates information about the first surgical instrument and information about the second surgical instrument, and
the training control unit is configured to overlay the first virtual surgical instrument and the second virtual surgical instrument on the surgical video of the first type.

7. The training device according to claim 1, wherein
the surgical video is a surgical video of a second type which does not include a graphical user interface that indicates information about the first surgical instrument and information about the second surgical instrument, and
the training control unit is configured to overlay the first virtual surgical instrument and the second virtual surgical instrument and a graphical user interface that indicates information about the first virtual surgical instrument and information about the second virtual surgical instrument, on the surgical video of the second type.

8. The training device according to claim 2, wherein
the training control unit is configured to display on the display the selection screen such that one of a first type surgical video which includes a graphical user interface that indicates information about the first surgical instrument and information about the second surgical instrument and a second type surgical video which does not include the graphical user interface is selectable through the selection screen.

9. The training device according to claim 1, wherein
the training control unit is configured to the display the first virtual surgical instrument and the second virtual surgical instrument on the surgical video on the display such that the displayed first virtual surgical instrument and the displayed second virtual surgical instrument are moved in response to the operation on the first input device and the operation on the second input device of the doctor side control apparatus, which is to be used during the surgery to operate the patient side apparatus.

10. A training system comprising:
a first terminal device provided in a first facility in which a first surgical system is installed, wherein the first surgical system includes: a first patient side apparatus and a first doctor side control apparatus configured to operate the first patient side apparatus; and a training control device configured to display on a first display of the first doctor side control apparatus a surgical video with overlaying a first virtual surgical instrument and a second virtual surgical instrument on the displayed surgical video;
a second terminal device provided in a second facility in which a second surgical system including a second patient side apparatus and a second doctor side control apparatus configured to operate the second patient side apparatus is installed;
a server provided in a third facility different from the first facility and the second facility and configured to communicate with the first terminal device and the second terminal device; and
a storage device provided in the third facility and configured to store therein surgical videos, wherein
the storage device is configured to receive a first surgical video, which is a video of surgery performed using the second surgical system of the second facility, from the second terminal device and store therein the first surgical video.

11. The training system according to claim 10, wherein
the server is configured, in response to an upload request from the second terminal device for uploading the first surgical video, to receive the first surgical video from the second terminal device and store the first surgical video in the storage device.

12. The training system according to claim 10, wherein
the server is configured, in response to a download request from the first terminal device for downloading the first surgical video, to permit the first terminal device to download the first surgical video stored in the storage device.

13. The training system according to claim 11, wherein
the server is configured, in response to a download request from the first terminal device for downloading the first surgical video, to permit the first terminal device to download the first surgical video stored in the storage device.

14. The training system according to claim 10, wherein
the first patient side apparatus includes a first robot arm to which a first surgical instrument is attached and a second robot arm to which a second surgical instrument is attached,
the first doctor side control apparatus includes a first input device configured to operate the first robot arm, a second input device configured to operate the second robot arm, and the first display, and
the training control device is configured to move the displayed first virtual surgical instrument in response to operation on the first input device and move the displayed second virtual surgical instrument in response to operation on the second input device.

15. The training system according to claim 14, wherein
the second patient side apparatus includes a third robot arm to which a third surgical instrument is attached and a fourth robot arm to which a fourth surgical instrument is attached, and
the second doctor side control apparatus includes a third input device configured to operate the third robot arm, a fourth input device configured to operate the fourth robot arm, and a second display.

16. The training system according to claim 10, wherein
the training control device is configured to overlay the first virtual surgical instrument and the second virtual surgical instrument that are displayed in thin color or translucent on the surgical video.

17. A training control device to be used for training for surgery using a doctor side control apparatus that includes a first input device configured to operate a first robot arm to which a first surgical instrument is attached; a second input device configured to operate a second robot arm to which a second surgical instrument is attached; and a display, the training control device comprising:
one or more processors configured with a program such that the training control device is configured to display on the display a surgical video with overlaying a first virtual surgical instrument and a second virtual surgical instrument on the displayed surgical video, in such a manner that the first virtual surgical instrument moves in response to operation on the first input device and the second virtual surgical instrument moves in response to operation on the second input device.

18. The training control device according to claim 17, wherein
the training control device is configured to overlay the first virtual surgical instrument and the second virtual surgical instrument that are displayed in thin color or translucent on the surgical video.

19. The training control device according to claim 17, wherein
the surgical video is a first type surgical video which includes a graphical user interface that indicates information about the first surgical instrument and information about the second surgical instrument.

20. The training control device according to claim 17, wherein
the surgical video is a second type surgical video which does not include a graphical user interface that indicates information about the first surgical instrument and information about the second surgical instrument, and
the training control device is configured to overlay the first virtual surgical instrument and the second virtual surgical instrument and a graphical user interface that indicates information about the first virtual surgical instrument and information about the second virtual surgical instrument, on the second type surgical video.
